(19) United States
(12) Patent Application Publication
(10) Pub. No.: US 2010/0178334 A1
(43) Pub. Date: Jul. 15, 2010

(54) Oral Pharmaceutical Dosage Form Comprising as Active Ingredients a Proton Pump Inhibitor together with Acetyl Salicyclic Acid

(76) Inventors: Dick Johansson, Molndal (SE); Lena Nilsson, Molndal (SE); Lars-Erik Svedberg, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 12/094,519

(22) PCT Filed: Nov. 28, 2006
(86) PCT No.: PCT/SE06/01349

§ 371 Date: May 21, 2008

Related U.S. Application Data
(60) Provisional application No. 60/740,981, filed on Nov. 30, 2005.
(60) Provisional application No. 60/818,886, filed on Jul. 6, 2006.

(51) Int. Cl. A61K 31/616; A61K 9/00; A61K 9/20; A61K 9/48; A61P 1/00; A61P 9/10; A61P 7/02

(52) U.S. Cl. 424/452; 424/400; 514/159; 424/464; 514/161

(57) ABSTRACT

The present invention relates to an oral pharmaceutical preparation for use in the prevention and/or reduction of gastrointestinal complications associated with the use of acetyl salicylic acid. The present preparation comprises a fixed oral dosage form comprising a proton pump inhibitor in combination with acetyl salicylic acid. Furthermore, the present invention refers to a method for the manufacture thereof and the use thereof in medicine. The present invention also relates to a specific combination comprising esomeprazole, or an alkaline salt thereof or a hydrated form of any one of them, and acetyl salicylic acid for use as a medicament for the prevention of thromboembolic vascular events, such as myocardial infarction or stroke, and for the prevention and/or reduction of gastrointestinal complications associated with the use of acetyl salicylic acid.

### FIELD OF THE INVENTION

The present invention relates to an oral pharmaceutical preparation for use in the prevention and/or reduction of gastrointestinal complications associated with acetyl salicylic acid treatment. The present preparation comprises a fixed oral dosage form comprising a proton pump inhibitor (hereinafter also referred to as a PPI, i.e. a proton pump inhibitor) in combination with acetyl salicylic acid (hereinafter also referred to as ASA) or a derivative thereof. Furthermore, the present invention refers to a method for the manufacture thereof and the use thereof in medicine.

The present invention also relates to a specific combination comprising esomeprazole, or an alkaline salt thereof or a hydrated form of any one of them, and acetyl salicylic acid in an oral fixed combination dosage form comprising a group of separate physical units comprising esomeprazole, or an alkaline salt thereof or a hydrated form of any one of them, and one or more other separate physical units comprising ASA or a derivative thereof for use as a medicament for the prevention of thromboembolic vascular events, such as myocardial infarction or stroke, the risk of which is increased in the elderly population and further prevention and/or reduction of gastrointestinal complications associated with acetyl salicylic acid (ASA) treatment.

### BACKGROUND OF THE INVENTION

Acetyl salicylic acid (ASA) is one of the most commonly prescribed and used drugs worldwide. Its use in prevention of thromboembolic vascular events, such as myocardial infarction or stroke have been described in “Collaborative overview of randomised trials of antiplatelet therapy Prevention of death, myocardial infarction, and stroke by prolonged antiplatelet therapy in various categories of patients.” [British Medical Journal 1994, 308, p. 81-106, by Antiplatelets triallists collaboration]. Despite the therapeutic benefits, its use is frequently limited by an increased risk of gastrointestinal side effects, mainly upper gastrointestinal side effects like peptic ulceration and dyspeptic symptoms. The relative risk of developing an ulcer complication like bleeding from the stomach or the duodenum is increased by all studied doses of ASA. A peptic ulcer always precedes a peptic ulcer bleed. Even a daily dose as low as 75 mg doubles this risk (Weil et al BMJ 1995:310; 827-830). Epidemiological data from the UK indicate that 18% of hospital admissions due to adverse drug reactions are caused to ASA (Pirmohamed et al BMJ 2004:329; 15-19). Therefore, therapies that avoid gastrointestinal side effect caused by ASA are requested.

The most promising solution to the problem of healing and preventing ASA associated upper gastrointestinal side-effects like ulcers and dyspeptic symptoms in patients with a need for continuous treatment is to combine the ASA treatment with an anti-ulcer drug is approved for the healing and/or prophylaxis of ASA associated gastrointestinal side-effects such as prostaglandin analogues, H₂-receptor antagonists or proton pump inhibitors.

“Schutzwirkung von Omeprazol gegenüber niedrig dosierter Acetylsalicylsäure” by Simon et al in Arzneimittel-Forschung, 1995 vol. 45 no. 6, p. 701-3, reports that concomitant administration of omeprazole for patients treated with ASA was found to reduce gastroduodenal lesions evoked by ASA.

In “Untersuchungen zur Schutzwirkung von Lanzoprazol auf die menschlische Magenschleimhaut gegenüber niedrig dosierter Acetylsalicylsäure” by Miller et al in Arzneimittel-Forschung, 1997 vol. 47 no. 6, p. 758-60, it was reported that concomitant administration of either lansoprazole or ranitidine for patients treated with ASA was found to reduce damages on the mucosa caused by ASA.

Established risk factors for developing ASA associated upper gastrointestinal side effects and complications are for instance high age, previous peptic ulcer and/or bleeding, high dose of ASA, co-therapy with other antithrombotic drugs, anticoagulants or Nonsteroidal Anti-inflammatory Drugs (NSAIDs). This means that for example, fragile and elderly patients tolerating a complication like bleeding or perforation badly should receive prophylactic treatment in connection with their ASA treatment.

This has for instance been suggested by A. Lanas in Digestive and Liver Disease, 2004, 36, p. 655-7.

Low-dose ASA is mainly used for the prevention of thromboembolic vascular events, such as myocardial infarction or stroke, the risk of which is increased in the elderly population. Compliance with treatment is especially important in elderly and fragile patients, who have the highest risk of developing a life-threatening complication to ASA treatment like bleeding or perforation. The importance of compliance is further supported by the finding, that peptic ulcers associated with ASA treatment are often asymptomatic until the event.

In proposed therapies comprising ASA and a proton pump inhibitor, the different active substances often are administered separately, as presented in “Clopidogrel versus Aspirin and Esomprazole to prevent recurrent bleeding.” in New England Journal of Medicine, 2005, 352, p. 238-44. It is well known that patient compliance is a main factor in receiving a good result in medical treatments. Therefore, administration of two or even more different tablets/capsules to the patient is not convenient or satisfactory to achieve the most optimal results.

In US 2005/0227949 A1 it is presented that a combination of an NSAID and a histamine H2-receptor antagonist is an effective treatment against viral and bacterial infections. Among the particularly preferred H2-histamine receptor antagonist is included omeprazole and esomeprazole. A kit comprising the compounds is claimed among other things. No fixed unit dosage form is disclosed.

WO 97/25064 describes an oral pharmaceutical dosage form comprising an acid susceptible proton pump inhibitor and one or more NSAIDs in a fixed formulation, wherein the proton pump inhibitor is protected by an enteric coating layer. The fixed formulation is in the form of an enteric coating layered tablet, a capsule or a multiple unit tableted dosage form. The multiple unit dosage forms are most preferred.

Some proton pump inhibitors are susceptible to degradation in acid reacting and neutral media. In respect of the stability properties, it is obvious that when one of the active substances being a acid susceptible proton pump inhibitor it must be protected from contact with acidic gastric juice by an enteric coating layer. There are different enteric coating layered preparations of proton pump inhibitors described in the prior art, see for example U.S. Pat. No. 4,786,505 (AB Hässle) comprising omeprazole.

US 2002/0155153 A1 discloses a fixed unit dosage form which can as one alternative be a capsule filled with more than one pharmaceutically active compound. The active compounds are preferably an acid susceptible proton pump inhibitor in combination with one or more NSAIDs and wherein at least the proton pump inhibitor is protected by an enteric coating layer.

US 2003/0069255 A1, now U.S. Pat. No. 6,926,907, discloses a single, coordinated, unit-dose product that combines an agent that actively raises intragastric pH, and an NSAID specially formulated to be released in a coordinated way. The figures show that the NSAID is situated inside an enteric coating while the agent that actively raises intragastric pH is located outside/on the enteric coat.

U.S. Pat. No. 6,554,556 B1 presents an invention that is directed to a solid oral dosage form comprising an NSAID extended release tablet and an enterically coated proton-pump inhibitor prepared without applying a separating layer between the proton pump inhibitor and the enteric coat.

US 2002/0051814 A1, now U.S. Pat. No. 7,029,701 B2, is directed to formulations having omeprazole and aspirin comprised in the same core and further some kind of coating around said core.

FR 2845917 relates to a pharmaceutical combination comprising tenatoprazole and an NSAID or COX-2 inhibitor.

Another patent application, US 2004/0121004 A1, presents a fixed unit dosage firm for an NSAID, a proton pump inhibitor and a buffer. The dosage forms are not enteric coated.

A further patent application that discloses a fixed unit dosage form which is not enteric coated, is US 2005/0147675 A1. This reference discloses a fast dissolving tablet comprising ASA and esomeprazole.

### OUTLINE OF THE INVENTION

The present invention relates to an oral pharmaceutical dosage form comprising a proton pump inhibitor together with acetyl salicylic acid and optionally pharmaceutically acceptable excipients, characterized in that the dosage form is in the form of an oral fixed combination dosage form comprising a group of separate physical units comprising a proton pump inhibitor and one or more other separate physical units comprising acetyl salicylic acid or a derivative thereof.

In the present invention, the dosage form is a capsule formulation, multiple unit tablet formulation or sachet formulation, which will simplify the regimen and improve the patient compliance and which will also provide a good stability to the active substances during long-term storage.

The dosage forms according to the invention are suitable to be used especially for the prevention of thromboembolic vascular events, such as myocardial infarction or stroke, the risk of which is increased in the elderly population and further the prevention and/or reduction of gastrointestinal complications associated with acetyl salicylic acid (ASA) treatment.

### DESCRIPTION OF THE INVENTION

### Embodiments of the Invention

A first embodiment of the present invention relates to an oral pharmaceutical dosage form comprising as active ingredients an acid susceptible proton pump inhibitor (PPI) together with acetyl salicylic acid (ASA) or a derivative thereof and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl salicylic acid or a derivative thereof, and wherein at least the proton pump inhibitor is protected by an enteric coating layer.

In a second embodiment of the present invention the oral pharmaceutical dosage form is comprising an acid susceptible proton pump inhibitor together with acetyl salicylic acid and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl salicylic acid or a derivative thereof, and wherein the proton pump inhibitor is protected by an enteric coating layer and the acetyl salicylic acid or a derivative thereof is not enteric coated.

In a third embodiment of the present invention the oral pharmaceutical dosage form is comprising an acid susceptible proton pump inhibitor together with acetyl salicylic acid or a derivative thereof and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl salicylic acid or a derivative thereof, and wherein the proton pump inhibitor is protected by an enteric coating layer and the acetyl salicylic acid or a derivative thereof is not enteric coated and further is present in an immediate release form.

A fourth embodiment of the invention is directed to an oral pharmaceutical dosage form which is comprising an acid susceptible proton pump inhibitor together with acetyl salicylic acid or a derivative thereof and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl to salicylic acid or a derivative thereof, and wherein the proton pump inhibitor comprising units are protected by an enteric coating layer and the unit comprising acetyl salicylic acid or a derivative thereof is compressed to a tablet and furthermore not is enteric coated.

A lift embodiment of the invention is directed to an oral pharmaceutical dosage form which is comprising an acid susceptible proton pump inhibitor together with acetyl salicylic acid or a derivative thereof, and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl salicylic acid or a derivative thereof, and wherein the units comprising the proton pump inhibitor are protected by an enteric coating layer and the unit comprising the acetyl salicylic acid or a derivative thereof is mildly compressed to a plug and furthermore not is enteric coated. The mild compression of ASA is beneficial for its stability and dissolution rate.

In one special embodiment of the invention, the mildly compressed plug of ASA has a friability as measured for tablets in US Pharmacopoeia 24, official from 1 Jan. 2000, in the range of 2%-50% (w/w), preferably 2%-30% (w/w) and more preferably 2-10% (w/w).

In another special embodiment of the invention, the mildly compressed plug of ASA has a friability as measured for tablets in US Pharmacopoeia 24, official from 1 Jan. 2000, in the range of 4%-50% (w/w), preferably 4%-30% (w/w) and more preferably 4-10% (w/w).

In a further special embodiment of the invention, the mildly compressed plug of ASA has a friability as measured for tablets in US Pharmacopoeia 24, official from 1 Jan. 2000, in the range of 6%-50% (w/w), preferably 6%-30% (w/w) and more preferably 6-10% (w/w).

### Terms Used;

The physical units, when used as a starting material for coating, are also referred to as “cores”, or as “core material”.

The term “dosage form” as used herein, is limited to capsule, tablet, “multiple unit tablet” (see p. 22) or sachet.

Thus the term “fixed combination dosage form” in the present invention is excluding a blister pack arrangement comprising separate dosage forms of PPI and ASA respectively, e.g. one capsule or tablet comprising the acid susceptible proton pump inhibitor and another capsule or tablet comprising the acetyl salicylic acid, packed together. This does not exclude that it is envisaged to pack the dosage forms of the invention in a blister pack cartridge.

The term “unit(s)”, as used herein, is intended to include “pellet(s)”, “granule(s)”, “bead(s)”, “mildly compacted plug(s)” and “tablet(s)”.

The term “tablet” is the normal, meaning any compressed tablet, which also fulfills the requirement regarding friability being less than 1% (w/w), as measured and required for tablets in US Pharmacopoeia 24, official from 1 Jan. 2000.

The term “mildly compacted plug” considers a material that have been compressed into a unit form like e.g. a tablet, but not enough compressed to fulfill the requirement of friability for tablets in US Pharmacopoeia 24, official from 1 Jan. 2000. The mildly compacted plugs are having a friability as measured for tablets, according to US Pharmacopoeia 24, official from 1 Jan. 2000, being 2% (w/w) or more. In special embodiments the friability is a range which might be situated starting from 2% (w/w) or above and upwards.

The term “gastrointestinal complications”, as used herein, is intended to include ulcer in the stomach or duodenum, complications to said ulcers, such as bleeding, perforation and/or obstruction, and dyspeptic symptoms, such as epigastric pain and/or discomfort.

The term “prevention”, as used herein, also includes the inhibition of “gastrointestinal complications”. The term “reduction” as used herein, is intended to also include the risk is reduction of “gastrointestinal complications”.

The term “ASA”, as used herein, is an abbreviation of acetyl salicylic acid.

The term “PPI”, as used herein, is an abbreviation of proton pump inhibitor, and thus encompasses esomeprazole, or an alkaline salt thereof or a hydrated form of any one of them, as well as omeprazole, or an alkaline salt thereof or a hydrated form of any one of them.

The expressions “low dose acetyl salicylic acid” or “low dose ASA”, as used herein, is in one embodiment defined as doses in the range of 10 mg to 500 mg of ASA. In another embodiment it is defined as doses in the range of 25 mg to 450 mg of ASA. In a further embodiment it is defined as doses in the range of 60 mg to 350 mg of ASA.

### Active Ingredients;

The acid susceptible proton pump inhibitors suitable for the present invention are H⁺K⁺-ATPase inhibitors and they are selected from:

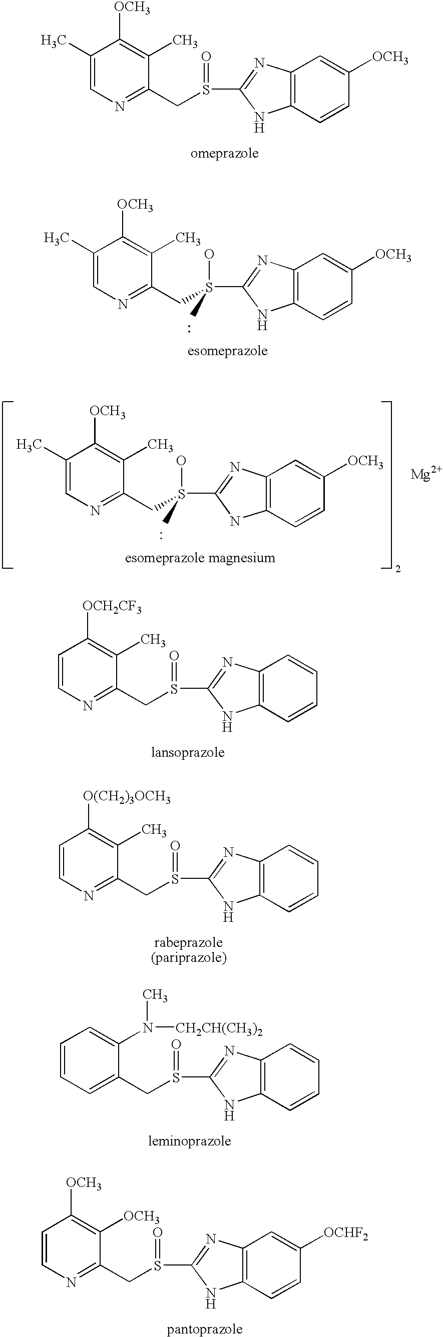

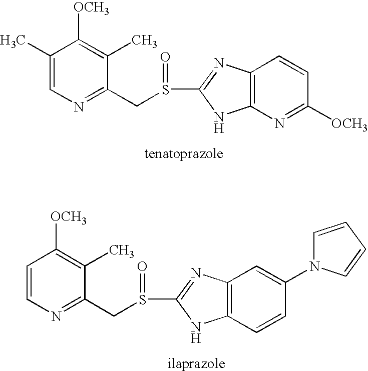

The acid susceptible proton pump inhibitors used in the dosage form of the present invention may be used in their neutral form or in the form of a pharmaceutically acceptable salt such as an alkaline salt selected from any one of their Mg²⁺, Ca²⁺, Na⁺, K⁺, Li⁺ or TBA (tert-butyl ammonium) salts. Further a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof, such as for instance hydrates. The above-listed compounds can also be used in their tautomeric form. Also included in the present invention are derivatives of the compounds listed above which have the biological function of the compounds listed, such as prodrugs.

Proton pump inhibitors are for example disclosed in EP-A1-0005129, EP-A1-174 726, EP-A1-166 287, GB 2 163 747 and WO90/06925, WO91/19711, WO91/19712, WO95/01977, WO98/54171 and WO94/27988.

The acetyl salicylic acid (ASA) can be selected from its free acid form, derivatives thereof or any other possible forms, for example, but not limiting to scope of the present invention, acetyl salicylic amid or acetyl salicylic complex(s).

In a further special embodiment of the present invention the acetyl salicylic acid is in its free acid form. In another further special embodiment of the present invention the acetyl salicylic acid is present as acetyl salicylic amid or acetyl salicylic complex(s) like e.g. a cyclodextrin complex.

Anyone of the different embodiments of ASA can be combined with anyone of the earlier presented embodiments of the oral pharmaceutical dosage form of the invention.

According to one embodiment of the invention, the acid susceptible PPI is omeprazole or an alkaline salt thereof or the acid susceptible PPI is esomeprazole, an alkaline salt thereof or a hydrate form of any one of them.

According to another embodiment of the invention, the acid susceptible PPI is omeprazole or an alkaline salt thereof.

According to yet another embodiment of the present invention the acid susceptible PPI is esomeprazole, an alkaline salt thereof or a hydrate form of any one of them.

According to a further embodiment of the present invention the acid susceptible PPI is lansoprazole or a pharmaceutically acceptable salt thereof or a single enantiomer of either one of them.

In another embodiment of the present invention the acid susceptible PPI is pantoprazole or a pharmaceutically acceptable salt thereof or a single enantiomer of either one of them.

In yet another embodiment of the present invention, the acid susceptible PPI is rabeprazole or a pharmaceutically acceptable salt thereof or a single enantiomer of either one of them.

In a further embodiment of the present invention, the acid susceptible PPI is ilaprazole or a pharmaceutically acceptable salt thereof or a single enantiomer of either one of them.

In yet a further embodiment of the present invention, the acid susceptible PPI is tenatoprazole or a pharmaceutically acceptable salt thereof or a single enantiomer of either one of them.

Anyone of the different embodiments of acid susceptible PPI can be combined with anyone of the earlier presented embodiments of ASA in anyone of the earlier presented embodiments of the oral pharmaceutical dosage form of the invention.

An active ingredient combination especially foreseen to be included in anyone of the earlier presented embodiments of the oral pharmaceutical dosage form is esomeprazole, an alkaline salt thereof or a hydrate form of any one of them and the acetyl salicylic acid is in its free acid form.

Another active ingredient combination especially foreseen to be included in anyone of the earlier presented embodiments of the oral pharmaceutical dosage form is omeprazole, an alkaline salt thereof or a hydrate form of any one of them and the acetyl salicylic acid is in its free acid form.

### Core Material

The core material for the individually enteric coating layered units can be constituted according to different principles. Seeds layered with the proton pump inhibitor, optionally mixed with alkaline substances, can be used as the core material for the further processing.

The seeds which are to be layered with the proton pump inhibitor may be water insoluble seeds comprising different oxides, celluloses, organic polymers and other materials, alone or in mixtures. The seeds may also be water-soluble seeds comprising different inorganic salts, sugars, non-pareils and other materials, alone or in mixtures. Further, the seeds may comprise the proton pump inhibitor in the form of crystals, agglomerates, compacts etc. The size of the seeds is not essential for the present invention but may vary from approximately 0.1 to 2 mm. In a preferred embodiment of the invention the average diameter of the seeds is from 0.1 mm up to 1.0 mm. The seeds layered with the proton pump inhibitor are produced either by powder or solution/suspension layering. Granulation or spray coating layering equipment may be used.

Before the seeds are layered, the proton pump inhibitor may be mixed with further components. Such components can be binders, surfactants, fillers, disintegrating agents, alkaline additives and/or other pharmaceutically acceptable ingredients, alone or in mixtures. The binders are for example polymers such as hydroxypropyl methylcellulose (HPMC), hydroxypropyl cellulose (HPC), carboxymethylcellulose sodium, polyvinyl pyrrolidone (PVP), or sugars, starches or other pharmaceutically acceptable substances with cohesive properties. Suitable surfactants are found in the groups of pharmaceutically acceptable non-ionic or ionic surfactants such as for instance sodium lauryl sulfate.

Alternatively, the proton pump inhibitor optionally mixed with alkaline substances and further mixed with suitable constituents can be formulated into a core material. Extrusion/spheronization, balling or compression utilizing conventional process equipment may produce said core material. The size of the formulated core material is in one embodiment of the invention approximately from 0.1 mm to 4 mm in diameter, and in another embodiment of the invention from 0.1 mm to 2 mm in diameter. The manufactured core material can further be layered with additional ingredients comprising the proton pump inhibitor and/or be used for further processing.

The proton pump inhibitor is mixed with pharmaceutical constituents to obtain suitable handling and processing properties and a suitable concentration of the proton pump inhibitor in the final preparation. Pharmaceutical constituents such as fillers, binders, lubricants, disintegrating agents, surfactants and other pharmaceutically acceptable additives may be used.

Further, the proton pump inhibitor may also be mixed with an alkaline, pharmaceutically acceptable substance (or substances). Such substances can be chosen among, but are not restricted to, substances such as the sodium, potassium, calcium, magnesium and aluminium salts of phosphoric acid, carbonic acid, citric acid or other suitable weak inorganic or organic acids; aluminium hydroxide/sodium bicarbonate co precipitate; substances normally used in antacid preparations such as aluminium, calcium and magnesium hydroxides; magnesium oxide or composite substances, such as Al₂O₃.6MgO.CO₂.12H₂O, (Mg₆Al₂(OH)₁₆CO₃.4H₂O), MgO.Al₂O₃.2SiO₂.nH₂O or similar compounds; organic pH-buffering substances such as trihydroxymethylaminomethane, basic amino acids and their salts or other similar, pharmaceutically acceptable pH-buffering substances.

Alternatively, the aforementioned core material can be prepared by using spray drying or spray congealing technique.

### Enteric Coating Layer(s)

Before applying the enteric coating layer(s) onto the core material in the form of individual units, the units may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of proton pump inhibitor should be water soluble or rapidly disintegrating in water.

The separating layer(s) can be applied to the core material by coating or layering procedures in suitable equipments, such as coating pan, coating granulator or in a fluidized bed apparatus using water and/or organic solvents for the coating process. As an alternative, the separating layer(s) can be applied to the core material by using powder coating technique. The materials for the separating layers are pharmaceutically acceptable compounds selected from any one of sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethyl cellulose sodium, water soluble salts of enteric coating polymers and others, used alone or in mixtures. Additives such as plasticizers, colorants, pigments, fillers anti-tacking and anti-static agents (such as magnesium stearate, titanium dioxide, talc) and other additives may also be included into the separating layer(s).

When the optional separating layer is applied to the core material, it may constitute a variable thickness. The maximum thickness of the separating layer(s) is normally only limited by processing conditions. The separating layer may serve as a diffusion barrier and it may also act as a pH-buffering zone. The pH-buffering properties of the separating layer(s) can be further strengthened by introducing into the layer(s) substances chosen from a group of compounds usually used in antacid formulations such as, for instance, magnesium oxide, hydroxide or carbonate, aluminium or calcium hydroxide, carbonate or silicate; composite aluminium/magnesium compounds such as Al₂O₃.6MgO.CO₂.12H₂O, (Mg₆Al₂(OH)₁₆CO₃.4H₂O), MgO.Al₂O₃.2SiO₂.nH₂O, aluminium hydroxide/sodium bicarbonate coprecipitate or similar compounds; or other pharmaceutically acceptable pH-buffering compounds such as, for instance the sodium, potassium, calcium, magnesium and aluminium salts of phosphoric, carbonic, citric or other suitable, weak, inorganic or organic acids; or suitable organic bases, including basic amino acids and salts thereof. Talc or other compounds may also be added to increase the thickness of the layer(s) and thereby strengthen the diffusion barrier. The optionally applied separating layer(s) is not essential to for the invention. However, the separating layer(s) may improve the chemical stability of the active substance and/or the physical properties of the claimed oral fixed dosage form.

Alternatively, the separating layer may be formed in situ by a reaction between an enteric coating polymer layer applied on the core material and an alkaline reacting compound in the core material. Thus, the separating layer formed comprises a water soluble salt formed between the enteric coating layer polymer(s) and an alkaline reacting compound, which is in the position to form a salt.

One or more enteric coating layers are applied onto the core material or onto the core material covered with separating layer(s) by using a suitable coating technique. The enteric coating layer material may be dispersed or dissolved in either water or in suitable organic solvents or suitable mixtures of water plus solvent when applicable, like e.g. water plus ethanol in certain proportions can be used to dissolve hydroxypropyl methylcellulose phthalate. As enteric coating layer polymers one or more, separately or in combination, of as the following can be used, e.g. solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable enteric coating polymer(s).

The enteric coating layers may contain pharmaceutically acceptable plasticizers to obtain the desired mechanical properties, such as flexibility and hardness of the enteric coating layers. Such plasticizers are selected from e.g. triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to selected enteric coating layer polymer(s), selected plasticizer(s) and the applied amount of said polymer(s), in such a way that the mechanical properties, i.e. flexibility and hardness of the enteric coating layer(s) fulfill the desired requirements. The amount of plasticizer is usually above 10% by weight of the enteric coating layer polymer(s), alternatively 15-50%, or alternatively 20-50%. Additives such as dispersants, colorants, pigments polymers e.g. poly (ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. To protect the acid susceptible substance, the proton pump inhibitor, and to obtain an acceptable acid resistance of the dosage form according to the invention, the enteric coating layer(s) constitutes a thickness of approximately at least 10 μm or alternatively more than 20 μm. The maximum thickness of the applied enteric coating is normally only limited by processing conditions and the desired dissolution profile. In one embodiment of the invention the enteric coating layer thickness is in the range of 15-45 micron. In a preferred embodiment of the invention the enteric coating layer thickness is in the range of 20-35 micron.

### Over-Coating Layer

Units comprising either proton pump inhibitor or ASA and covered with enteric coating layer(s) may further be covered with one or more over-coating layer(s). The over-coating layer(s) should be water soluble or rapidly disintegrating in water. The over-coating layer(s) can be applied to the enteric coating layered units by coating or layering procedures in suitable equipments, such as coating pan, coating granulator or in a fluidized bed apparatus using water and/or organic solvents for the coating or layering process. The materials for over-coating layers are chosen among pharmaceutically acceptable compounds selected from any one of sugar, polyethylene glycol, polyvinyl pyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking and anti-static agents (such as magnesium stearate, titanium dioxide and talc) and other additives may also be included into the over-coating layer(s). Said over-coating layer may further prevent potential agglomeration of enteric coating layered units. The maximum thickness of the applied over-coating layer(s) is normally limited by processing conditions and the desired dissolution profile.

In one embodiment of the present invention the proton pump inhibitor is protected by two layers, an enteric coating layer and a subcoating layer separating the enteric coating from the proton pump inhibitor.

For filling enteric coated units or overcoated enteric coated units into capsules, it is sometimes an advantage to admix a lubricant or a glidant. Such lubricants or glidants include Mg-Stearate, sodium stearyl fumarate, glyceryl behenate, talk and fumed silica, thereby not excluding the possibility to use other non-mentioned pharmaceutically acceptable lubricants or glidants.

In one embodiment of the invention the lubricant is Mg-Stearate.

In another embodiment of the invention the lubricant is sodium stearyl fumarate.

In a further embodiment of the invention the lubricant is glyceryl behenate.

### The Different Forms of Acetyl Salicylic Acid (ASA)

The ASA can be present in the following forms:
Powder of ASA (ASA-substance as such);
Agglomerates of ASA;
Spherical agglomerates of ASA;
Solid dispersions or solutions of ASA in polymers;
   These solid dispersions or solutions of may be accomplished by melting the dispersing/dissolving agent and adding the ASA, or by dissolving the dispersing/dissolving agent and ASA in a common solvent, where after the solvent is evaporated.
Cyclodextrin complexes of ASA (as powder);
   These complexes may comprise α-cyclodextrin, β-cyclodextrin, γ-cyclodextrin or derivates thereof such as e.g. β-hydroxypropyl cyclodextrin. The complexing cyclodextrin may be chosen to affect the release rate, for instance to give extended release (β-hydroxypropyl cyclodextrin) or immediate release (β-cyclodextrin).
Cyclodextrin complexes of ASA granulated together with pharmaceutical excipients;
   These complexes may comprise α-cyclodextrin, β-cyclodextrin, γ-cyclodextrin or derivates thereof such as e.g. β-hydroxypropyl cyclodextrin. The complexing cyclodextrin may be chosen to affect the release rate, for instance to give extended release (β-hydroxypropyl cyclodextrin) or immediate release (β-cyclodextrin).
Units for immediate release, comprising ASA together with pharmaceutical excipients;
Units for extended release, comprising ASA together with pharmaceutical excipients. These units may be constructed according to the hydrophilic gel matrix principle, hydrophobic matrix principle or diffusion membrane layered pellets/granules principle;
Units for enteric release (enteric coated granules or pellets), comprising ASA together with pharmaceutical excipients;
Units for pH-independent time delayed release ((not enteric coated) granules or pellets), comprising ASA together with pharmaceutical excipients;
Units comprising ASA together with effervescent pharmaceutical excipients for immediate release;
Units layered with an enteric coating layer, such as the enteric coating layer described above, comprising ASA;
Minitablets comprising ASA;
Coated Minitablets comprising ASA
Mildly compacted plug of ASA, which considers a material that have been compressed into a unit form like e.g. a tablet, with a friability that does not fulfill the requirement of friability for tablets in US Pharmacopoeia 24, official from 1 Jan. 2000 (requirement: less than 1%). See previously explanations.

### Process for Preparing the Claimed Fixed Dosage Form

The present invention also relates to a process for the manufacture of an oral fixed combination dosage form comprising an acid susceptible proton pump inhibitor and acetyl salicylic acid, characterized in that the proton pump inhibitor is prepared in the form of enteric coating layered units and that the units are mixed with acetyl salicylic acid and this mixture is optionally mixed with pharmaceutically acceptable excipients, and then the obtained mixture is filled into a capsule or a sachet. The acetyl salicylic acid can be in any of the forms disclosed above.

One embodiment of the present invention relates to a process for the manufacture of an oral fixed combination dosage form comprising an acid susceptible proton pump inhibitor and acetyl salicylic acid, characterized in that said proton pump inhibitor is prepared in the form of enteric coating layered units and that the units are filled into a capsule or a sachet together with one or more other separate physical units comprising acetyl salicylic acid optionally mixed with pharmaceutically acceptable excipients.

One example on a process for the manufacture of the present fixed dosage form, but which should not in any way limit the scope of the present invention, is to dry mix the PPI and ASA and then fill those active compounds into a capsule or sachet. The proton pump inhibitor is in the form of enteric coating layered units and the acetyl salicylic acid is in the form of units that may either be used as such or be in the form of modified release formulated units such as enteric coating layered units or in the form of units formulated to achieve an extended release e.g. by being coated with an extended release coating layer.

As another example of a manufacturing process, but which should not in anyway limit the scope of the present invention, is wet massed granulation. The acetyl salicylic acid is dry mixed with excipients, wherein one or more of the excipients optionally is a disintegrant. Suitable excipients for the acetyl salicylic acid granulation may be selected from any one of sodium starch glycolate, corn starch, crosslinked polyvinylpyrrolidone, low substituted hydroxypropyl cellulose, microcrystalline cellulose, mannitol, lactose and colloidal silicon dioxide anhydrous (Aerosil®).

The mixture is wet massed with a granulation liquid comprising a binder selected from any one of polyvinyl pyrrolidone, hydroxypropyl methyl cellulose, polyethylene glycol, hydroxypropyl cellulose and optionally one or more wetting agents, such as sodium lauryl sulphate, and a solvent such as purified water or a suitable alcohol or a mixture thereof. In one embodiment of the invention, the wet mass is dried to a loss on drying of less than 3% by weight. In another embodiment of the invention, the wet mass is dried to a loss on drying of less than 2% by weight.

After the drying the dry mass is milled to a suitable size for the granules, such as smaller than 4 mm, alternatively smaller than 1 mm.

The dry granules are then mixed with the proton pump inhibitor, which PPI is in the form of enteric coating layered units, and then filled into a capsule or a sachet or compressed, optionally together with suitable pharmaceutical excipients, to a “multiple unit tablet”.

In an alternative manufacturing process ASA, or granules of ASA and optionally pharmaceutical excipients, are compressed into a mildly compacted plug (definition according to above) and filled into a capsule; together with the PPI wherein the latter is in the form of enteric coating layered units.

The plug may be positioned in the lower part of the capsule, i.e. the body part, or in the upper part of the capsule, i.e. the cap. In both situations the plug is in tight connection to the inner walls of the capsule, restricting the free movement of PPI comprising units within the capsule. This is favourable for reducing intracapsular attrition.

The PPI comprising units may be positioned under the plug or on top of the plug, (in both situations within the capsule).

Thus, in one embodiment of the invention, the ASA comprising plug is positioned in the body part of the capsule in tight connection to the inner walls of the capsule and the PPI comprising units are positioned on top of the plug within the capsule.

In a further embodiment of the invention, the ASA comprising plug is positioned in the body part of the capsule in tight connection to the inner walls of the capsule and the PPI comprising units are positioned below the plug within the capsule.

In an even further embodiment of the invention, the ASA comprising plug is positioned in the cap (i.e. upper) part of the capsule in tight connection to the inner walls of the capsule cap and the PPI comprising units are positioned below the plug within the capsule body.

The acetyl salicylic acid may also be mixed with a gelling agent during the granulation, such as hydrophilic polymer(s) to obtain extended release. Suitable gelling hydrophilic polymers may be selected from any one of hydroxypropyl methylcellulose with a viscosity higher or equal to 50 mPas (cps), polyoxyethylene (polyethylene glycol) with a molecular weight above 50000 u, hydroxypropyl cellulose not including low-substituted hydroxypropyl cellulose, hydroxyethyl cellulose and xantan or combinations thereof.

The obtained units may also comprise suitable buffering substances.

### Capsule or Sachet Material

The capsule or sachet comprises any water-soluble or gastric soluble polymeric material, such as gelatin or hydroxypropyl methylcellulose. However, this list should however not be interpreted as exhaustive. The capsules or sachet may be produced by molding.

### Use of the Claimed Invention

The dosage forms according to the present invention are especially advantageous in the prevention and/or reduction of gastrointestinal complications caused by acetyl salicylic acid, for example in a continuous treatment with acetyl salicylic acid.

According to one embodiment of the present invention, the claimed dosage form has an amount of proton pump inhibitor in the range of from 5 to 300 mg and an amount of acetyl salicylic acid in the range of from 10 to 500 mg.

According to yet another embodiment the amount of proton pump inhibitor is in the range of from 10 to 200 mg or from 10 to 100 mg or from 10 to 80 mg. In an alternative embodiment of the present invention the amount of proton pump is selected from about: 5, 10, 20, 30, 40, 50, 60, 70, 80, 90 and 100 mg. According to yet another embodiment of the present invention, the amount of proton pump inhibitor is selected from 20, 40 and 80 mg.

In further embodiments of the present invention the amount of acetyl salicylic acid is in the range of from 25 to 450 mg, from 50 to 400, from 60 to 350 mg or from 75 to 325 mg. In an alternative embodiment of the present invention the amount of acetyl salicylic acid is selected from about: 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, and 325 mg, for example 81, 101, 124, 126, 181, 204, 301, 311 and 321.

In another embodiment of the present invention the oral fixed combination dosage form comprises 20 mg of esomeprazole and 325 mg of acetyl salicylic acid.

In a second other embodiment of the present invention the oral fixed combination dosage form comprises 20 mg of esomeprazole and 75 mg of acetyl salicylic acid.

In a third other embodiment of the present invention the oral fixed combination dosage faun comprises 40 mg of esomeprazole and 325 mg of acetyl salicylic acid.

In a fourth other embodiment of the present invention the oral fixed combination dosage form comprises 40 mg of esomeprazole and 75 mg of acetyl salicylic acid.

In a fifth other embodiment of the present invention the oral fixed combination dosage form comprises 20 mg of esomeprazole and 81 mg of acetyl salicylic acid.

In a sixth other embodiment of the present invention the oral fixed combination dosage faun comprises 40 mg of esomeprazole and 81 mg of acetyl salicylic acid.

The present invention also relates to a method for the prevention of thromboenabolic vascular events, such as myocardial infarction or stroke and the reduction and/or prevention of gastrointestinal complications associated with acetyl salicylic acid treatment, such as e.g. esophagitis associated with low dose ASA treatment, in mammals or man by administering to a mammals or man in need thereof a therapeutically effective dose of the claimed oral fixed combination dosage form. According to further embodiments of the present invention said complication is an upper gastrointestinal complication, a peptic ulcer in the stomach or a peptic ulcer in the duodenum. Upper gastrointestinal complications include bleeding, perforation and gastric outlet obstruction.

According to yet another embodiment of the present invention, the man is a patient of 60 years or older.

According to an alternative embodiment of the present invention the claimed method comprises administration of a capsule or a sachet comprising acetyl salicylic acid and proton pump inhibitor. The administration is either once or twice daily.

The present invention also relates to the use of a dosage form comprising a proton pump inhibitor and acetyl salicylic acid for the manufacture of a medicament for the prevention of thromboembolic vascular events, such as myocardial infarction or stroke, and for the prevention and/or reduction of gastrointestinal complications associated with acetyl salicylic acid treatment. According to further embodiments of the present invention, the complication is, as mentioned above, an upper gastrointestinal complication or is a peptic ulcer in the stomach or a peptic ulcer in the duodenum.

The present invention also relates to an oral pharmaceutical fixed combination dosage form comprising esomeprazole or an alkaline salt thereof or a hydrated form of any one of them and acetyl salicylic acid for the prevention of thromboembolic vascular events, such as myocardial infarction or stroke, and for the prevention and/or reduction of gastrointestinal complications associated with acetyl salicylic acid treatment. Any oral dosage form can be used for administration of this pharmaceutical combination, for instance a capsule, sachet, tablet or multiunit tablet, including effervescent forms thereof. However, this list should however not be interpreted as exhaustive.

An alternative embodiment of the present invention relates to a pharmaceutical oral fixed combination dosage form comprising esomeprazole or an alkaline salt thereof or a hydrated form of any one of them and acetyl salicylic acid, which dosage form is comprising a group of separate physical units comprising the acid susceptible proton pump inhibitor and one or more other separate physical units comprising the acetyl salicylic acid or a derivative thereof, and wherein at least the proton pump inhibitor is protected by an enteric coating layer, for the prevention of thromboembolic vascular events, such as myocardial infarction or stroke, and for the prevention and/or reduction of gastrointestinal complications associated with acetyl salicylic acid treatment.

In a further alternative embodiment of the invention, the unit (ASA comprising unit mentioned in the paragraph above) comprising the acetyl salicylic acid is compressed and used for the prevention of thromboembolic vascular events, such as myocardial infarction or stroke, and for the prevention and/or reduction of gastrointestinal complications associated with acetyl salicylic acid treatment.

In an even further alternative embodiment of the invention, the unit (ASA comprising unit mentioned in the penultimate paragraph above) comprising the acetyl salicylic acid is mildly compressed to a plug, and used for the prevention of thromboembolic vascular events, such as myocardial infarction or stroke, and for the prevention and/or reduction of gastrointestinal complications associated with acetyl salicylic acid treatment.

In one embodiment of the present invention, the claimed pharmaceutical combination has an amount esomeprazole or an alkaline salt thereof or a hydrated form of any one of them in the range of from 5 to 300 mg and an amount of acetyl salicylic acid of from 10 to 500 mg.

According to a further embodiment of the present invention, the amount of esomeprazole or an alkaline salt thereof or a hydrated form of any one of them is in the range of from 10 to 80 mg. According to yet another embodiment the amount of esomeprazole or an alkaline salt thereof or a hydrated form of any one of them is selected from 20, 40 or 80 mg.

In further embodiments of the present invention the amount of acetyl salicylic acid is in the range of from 25 to 450 mg, from 50 to 400, from 60 to 350 mg or from 75 to 325 mg. In an alternative embodiment of the present invention the amount of acetyl salicylic acid is selected from about: 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, and 325 mg, for example 81, 101, 124, 126, 181, 204, 301, 311 and 321.

A further embodiment of the present invention relates to a method for the prevention of thromboembolic vascular events, such as myocardial infarction or stroke, and for preventing and/or reducing gastrointestinal complications associated with acetyl salicylic acid treatment in mammals or man by administering to a mammals or man in need thereof the claimed pharmaceutical combination.

### EXAMPLES

The present invention is described in more detail by the following examples, which should not in any way limit the scope of the present invention.

### Example 1

Male or female Helicobacter pylori-negative patients ≧60 years, who had a moderate-to-high risk of developing gastroduodenal ulcers were included in this randomized, double-multicenter, placebo-controlled trial. Patients were randomized to receive either esomeprazole 20 mg (administered as esomeprazole magnesium, i.e. Nexium® owned by AstraZeneca AB) or placebo once daily for 26 weeks. The primary outcome variable was the presence of gastric and/or duodenal ulcers at endoscopy over the 26-week period. A total of 991 patients, all receiving ASA in doses varying between 75-325 mg/day (57.1% male, mean age 69.3 years, mean acetyl salicylic acid (ASA) dose 124.0 mg/day) were included in the intent-to-treat population. The cumulative proportion of patients without either gastric or duodenal ulcer at 26 weeks was 98.2% with esomeprazole, compared with 93.8% with placebo (life table estimates, p=0.0007). The incidence of gastric ulcers was lower in patients taking esomeprazole than in those taking placebo (1.2% vs. 3.8%), as was the incidence of duodenal ulcers (0.4% and 1.6% for esomeprazole and placebo, respectively). Eight patients (1.6%) had developed an ulcer, in the esomeprazole group by 6 months, compared with 27 patients (5.4%) in the placebo group. This corresponded to a relative reduction of developing an ulcer of 70% when taking esomeprazole rather than placebo. A total of 95.6% of patients treated with esomeprazole had no esophageal lesions at week 26, compared with 81.7% of patients treated with placebo (p<0.0001). The proportion of patients without esophageal lesions at 6 months was higher with esomeprazole than with placebo for patients with no lesions and for those with Los Angeles grade A lesions at baseline. Resolution of investigator-assessed upper gastrointestinal symptoms was higher with esomeprazole than with placebo for all symptoms. Esomeprazole was safe and well tolerated.

### Example 2

Capsule comprising Esomeprazole 20 mg and ASA granules 325 mg.

Principle: enteric coated pellets comprising Esomeprazole-Mg trihydrate corresponding to 20 mg Esomeprazole were manufactured and mixed with Mg-Stearate. This mixture and ASA granules were filled into hard gelatine capsules.

### Manufacturing of Enteric Coated Esomeprazole Pellets

### Core Material

| | |
|---|---|
| Sugar sphere seeds 0.25 to 0.35 mm approx. diameter | 300 g |

(Suspension for) Active Layer

| | | |
|---|---|---|
| Esomeprazole-Mg trihydrate | 445 | g |
| Hydroxypropyl methylcellulose | 67 | g |
| Polysorbate 80 | 9 | g |
| Purified water | 2100 | g |

(Suspension for) Subcoating layer

| | | |
|---|---|---|
| Hydroxypropyl cellulose | 90 | g |
| Talc | 340 | g |
| Magnesium stearate | 22 | g |
| Purified water | 3100 | g |

(Dispersion for) Enteric Coating Layer

| | | |
|---|---|---|
| Methacrylic acid copolymer type C, 30% dispersion | 1270 | g |
| Triethyl citrate | 38 | g |
| Mono- and diglycerides | 19 | g |
| Polysorbate 80 | 2 | g |
| Purified water | 500 | g |

Esomeprazole-Mg trihydrate was suspended in a water solution containing the dissolved binder hydroxypropyl methyl cellulose and the surfactant polysorbate 80. The suspension was sprayed onto sugar spheres seeds in a fluidized bed coating apparatus using bottom spray (Wurster) technique.

The prepared core material was covered with the subcoating layer in a fluid bed apparatus by spraying a hydroxypropyl cellulose solution containing suspended talc and magnesium stearate.

The enteric coating layer was sprayed as a water dispersion onto the subcoated pellets obtained above, in a fluid bed apparatus.

### Mixture of Enteric Coated Esomeprazole Pellets and Mg-Stearate.

Enteric coated pellets according to above was mixed with Mg-Stearate in the weight proportions given below;

| | |
|---|---|
| Esomeprazole gastro-resistant pellets | 100 |
| Magnesium stearate | 0.2 |

| Capsule filling | Per capsule | |
|---|---|---|
| Mixture of enteric coated Esomeprazole pellets | 86.2 | mg |
| and Mg-Stearate (acc. to above) | | |
| ASA granules* | 325 | mg |
| Hard gelatin capsule size 0 | 1 | piece |

*Rhodine ® 3118 ASA granules, Ba 0407231, from Rhodia France. The majority of the granules passes a sieve having apertures of 1000 micron and is retained on a sieve having apertures of 125 micron.

Capsules according to above was placed in plastic (High Density Poly Ethylene, also referred to as HDPE) bottles with desiccant, and checked for stability. The results obtained can be seen in the Table below;

| | | | % released | Sum degradation | Amount |
|---|---|---|---|---|---|
| | | | in pH 6.8 after | products, | degradation |
| | | | preexposure** | (%) of | of ASA. |
| Environment | Time | desiccant | Esomeprazole | Esomeprazole | (%) SA* |
| | 0 | | 93% | 0.2 | 0.3 |
| 40/75 | 2 weeks | 5 g | | 0.3 | NT |
| 40/75 | 4 weeks | 5 g | | 0.3 | NT |
| 30/75 | 3 months | 0.5 g | | 0.4 | NT |
| 25/60 | 6 months | 0.5 g | 93% | 0.4 | NT |

*SA = salicylic acid
NT = Not tested
**Dissolution of esomeprazole was measured in USP dissolution apparatus No 2 (paddle, 100 rpm) after preexposure in 300 ml 0.1 M HCl for 2 hrs, whereafter 700 ml of phosphate buffer was added giving a 1000 ml resulting testmedium having pH 6.8. After 30 minutes in pH 6.8 the released amount of nominal dose was measured.

### Example 3

Capsule comprising Esomeprazole 20 mg and ASA powder 325 mg.

Principle: enteric coated pellets comprising Esomeprazole-Mg trihydrate corresponding to 20 mg Esomeprazole were manufactured and mixed with Mg-Stearate, according to Ex. 2. This mixture and ASA powder were filled into hard gelatine capsules.

| Capsule filling | Per capsule | |
|---|---|---|
| Mixture of enteric coated Esomeprazole pellets | 86.2 | mg |
| and Mg-Stearate (acc. to Example 2, above) | | |
| ASA powder | 325 | mg |
| Hard gelatin capsule size 0 | 1 | piece |

Capsules according to above was placed in plastic (High Density Poly Ethylene, also referred to as HDPE) bottles with desiccant, and checked for stability. The results obtained can be seen in the Table below;

| | | | Sum | |
|---|---|---|---|---|
| | | | degradation | Amount |
| | | | products, | degradation |
| | | | (%) of | of ASA. |
| Environment | Time | desiccant | Esomeprazole | (%) SA |
| | 0 | | 0.2 | 0.2 |
| 25/60 | 3 months | 0.5 g | 0.2 | <0.1 |
| 25/60 | 6 months | 0.5 g | 0.2 | NT |

NT = Not tested

### Example 4

Capsule comprising Esomeprazole 20 mg and ASA (comprised in tablet) 75 mg.

Principle: enteric coated pellets comprising Esomprazole-Mg trihydrate corresponding to 20 mg Esomeprazole were manufactured and mixed with Mg-Stearate, according to Ex. 2. This mixture and ASA tablets were filled into hard gelatine capsules.

| Capsule filling | Per capsule | |
|---|---|---|
| Mixture of enteric coated Esomeprazole pellets | 86.2 | mg |
| and Mg-Stearate (acc. to Example 2, above) | | |
| ASA tablet comprising 75 mg ASA* | Approx. 97 | mg |
| Hard gelatin capsule size 1 | 1 | piece |

*Trombyl ®, Ba B 811A from Pfizer. Flat, hart-shaped uncoated tablets, approximazed size 6-7 mm in diameter, weight 97 mg (as average of 10 tablets).

Capsules according to above was placed in plastic (High Density Poly Ethylene, also referred to as HDPE) bottles with desiccant, and checked for stability. The results obtained can be seen in the Table below;

| | | | % released | Sum degradation | Amount |
|---|---|---|---|---|---|
| | | | in pH 6.8 after | products, | degradation |
| | | | preexposure** | (%) of | of ASA. |
| Environment | Time | desiccant | Esomeprazole | Esomeprazole | (%) SA |
| | 0 | | 93% | 0.2 | 2.3 |
| 40/75 | 1 month | 0.5 g | | 0.5 | 2.9 |
| 25/60 | 5 months | 0.5 g | 94% | 0.3 | NT |

*Dissolution of esomeprazole was measured in USP dissolution apparatus No 2 (paddle, 100 rpm) after preexposure in 300 ml 0.1 M HCl for 2 hrs, whereafter 700 ml of phosphate buffer was added giving a 1000 ml resulting testmedium having pH 6.8. After 30 minutes in pH 6.8 the released amount of nominal dose was measured.

### Example 5

Capsule comprising Esomeprazole 20 mg and ASA (comprised in enteric coated pellets) 100 mg.

Principle: enteric coated pellets comprising Esomeprazole-Mg trihydrate corresponding to 20 mg Esomeprazole were manufactured and mixed with Mg-Stearate, according to Ex. 2. This mixture and ASA enteric coated pellets were filled into hard gelatine capsules,

| Capsule filling | Per capsule | |
|---|---|---|
| Mixture of enteric coated Esomeprazole pellets | 86.2 | mg |
| and Mg-Stearate (acc. to Example 2, above) | | |
| ASA enteric coated pellets comprising 100 mg ASA* | 117.9 | mg |
| Hard gelatin capsule size 1 | 1 | piece |

*content of capsules “Astrix ®”, ba 298140, manufactured by Faulding & Co Ltd, Australia.

Capsules according to above was placed in plastic (High Density Poly Ethylene, also referred to as HDPE) bottles with desiccant, and checked for stability. The results obtained can be seen in the Table below;

| | | | % released | Sum degradation | Amount |
|---|---|---|---|---|---|
| | | | in pH 6.8 after | products, | degradation |
| | | | preexposure** | (%) of | of ASA. |
| Environment | Time | desiccant | Esomeprazole | Esomeprazole | (%) SA |
| | 0 | | 93% | 0.2 | 2.7 |
| 40/75 | 1 month | 0.5 g | | 0.3 | 3.9 |
| 25/60 | 5 months | 0.5 g | 95% | 0.2 | NT |

**Dissolution of esomeprazole was measured in USP dissolution apparatus No 2 (paddle, 100 rpm) after preexposure in 300 ml 0.1 M HCl for 2 hrs, whereafter 700 ml of phosphate buffer was added giving a 1000 ml resulting testmedium having pH 6.8. After 30 minutes in pH 6.8 the released amount of nominal dose was measured.

### Example 6

Capsule comprising Esomeprazole 20 mg and ASA granules 75 mg.

Principle: enteric coated pellets comprising Esomeprazole-Mg trihydrate corresponding to 20 mg Esomeprazole were manufactured and mixed with Mg-Stearate, according to Ex. 2. This mixture and a mildly compacted plug of ASA were filled into hard gelatine capsules.

### Manufacturing of Enteric Coated Esomeprazole Pellets

Was done according to Ex. 2.

### Mixture of Enteric Coated Esomeprazole Pellets and Mg-Stearate.

Enteric coated pellets according to above was mixed with Mg-Stearate in the weight proportions given below;

| | |
|---|---|
| Esomeprazole gastro-resistant pellets | 100 |
| Magnesium stearate | 0.2 |

| Capsule filling | Per capsule | |
|---|---|---|
| Mixture of enteric coated Esomeprazole pellets and | 86.2 | mg |
| Mg-Stearate (acc. to above) | | |
| ASA granules, compacted into a plug* | 75 | mg |
| Hard gelatin capsule size 2 | 1 | piece |

*Rhodine ® 3118 ASA granules, Ba FRH 0528131, from Rhodia France. The majority of the granules passes a sieve having apertures of 1000 micron and is retained on a sieve having apertures of 125 micron. The plug was positioned in the lower part of the capsule, i.e. the body part, in tight connection to the inner walls of the capsule.

Capsules according to above was packed in blister cartridges, having a three-layer film of PVC/Aclar®*/PVC and an Al-foil backing.

(*=Aclar® film is polychlorotrifluoroethylene film presently manufactured by Honeywell International Inc.)

Such capsules were also placed in plastic (High Density Poly Ethylene, also referred to as HDPE) bottles with desiccant, and checked for stability. The results obtained can be seen in the Table below;

| | | | Sum degradation | Amount |
|---|---|---|---|---|
| | | | products, | degradation |
| | | | (%) of | of ASA. |
| Environment | Time | desiccant | Esomeprazole | (%) SA* |
| | 0 | | 0.1 | NT |
| 40/75 | 3 months | 0.5 g | 0.7 | 0.1 |
| 30/75 | 3 months | 0.5 g | 0.1 | 0.1 |

*SA = salicylic acid
NT = Not tested

### Example 7

Tablet comprising Esomeprazole 20 mg and ASA 100 mg.

Principle: enteric coated pellets comprising Esomeprazole-Mg trihydrate corresponding to 20 mg Esomeprazole are prepared and overcoated with a layer of hydroxypropyl methyl cellulose, and then mixed with ASA granules and tablet excipients and compressed into multiple unit tablets.

### Manufacturing of Enteric Coated Esomeprazole Pellets

### Core Material

| | |
|---|---|
| Sugar sphere seeds 0.25 to 0.35 mm approx. diameter | 300 g |

(Suspension for) Active Layer

| | | |
|---|---|---|
| Esomeprazole-Mg trihydrate | 445 | g |
| Hydroxypropyl methylcellulose | 67 | g |
| Polysorbate 80 | 9 | g |
| Purified water | 2100 | g |

(Suspension for) Subcoating Layer

| | | |
|---|---|---|
| Hydroxypropyl cellulose | 90 | g |
| Talc | 340 | g |
| Magnesium stearate | 22 | g |
| Purified water | 3100 | g |

(Dispersion for) Enteric Coating Layer

| | | |
|---|---|---|
| Methacrylic acid copolymer type C, | 1270 | g |
| 30% dispersion | | |
| Triethyl citrate | 114 | g |
| Mono- and diglycerides | 19 | g |
| Polysorbate 80 | 2 | g |
| Purified water | 500 | g |

Esomeprazole-Mg trihydrate was suspended in a water solution containing the dissolved binder hydroxypropyl methyl cellulose and the surfactant polysorbate 80. The suspension was sprayed onto sugar spheres seeds in a fluidized bed coating apparatus using bottom spray (Wurster) technique.

The prepared core material was covered with the subcoating layer in a fluid bed apparatus by spraying a hydroxypropyl cellulose solution containing suspended talc and magnesium stearate.

The enteric coating layer was sprayed as a water dispersion onto the subcoated pellets obtained above, in a fluid bed apparatus.

(Solution for) Overcoating Layer

| | |
|---|---|
| Hydroxypropyl methyl cellulose 5-6 cps (mPas) | 90 g |
| Purified water | 2400 g |

The prepared enteric coated pellets from Example 2 are covered with the overcoating layer in a fluidized bed apparatus by spraying the hydroxypropyl methyl cellulose solution according to above onto them and drying when the spraying is completed.

The overcoated enteric coated Esomeprazole pellets are used for tableting;

| | Per 1000 | |
|---|---|---|
| Ingredients | tablets | |
| Overcoated enteric coated esomeprazole pellets | 103 | g |
| ASA granules* | 100 | g |
| Microcrystalline cellulose (Avicel PH 102) | 100 | g |
| Sodium Stearyl fumarate (Pruv ®) | 2.9 | g |
| Sum | 305.9 | g |

*example given, granules from Rhodia as in example 2.

The ingredients above are mixed in a laboratory mixer, type Kenwood for 3-4 minutes then compressed into tablets in a suitable tabletting machine, non-limiting example given is Korsch Pharmapress 106, using 9 mm circular biconvex punches, adjusting the average tablet weight to 306 mg/tablet.

1. An oral pharmaceutical dosage form comprising as active ingredients an acid susceptible proton pump inhibitor (PPI) together with acetyl salicylic acid (ASA) or a derivative thereof and optionally pharmaceutically acceptable excipients, characterized in that the dosage form is in the form of an oral fixed combination dosage form comprising a group of separate physical units comprising the acid susceptible proton pump inhibitor and one or more other separate physical units comprising the acetyl salicylic acid or a derivative thereof, and wherein at least the proton pump inhibitor is protected by an enteric coating layer.

2. A dosage form according to claim 1, wherein the proton pump inhibitor is protected by an enteric coating layer and the acetyl salicylic acid or a derivative thereof is not enteric coated.

3. A dosage form according to claim 2, wherein the acetyl salicylic acid or a derivative thereof further is present in an immediate release form.

4. A dosage form according to claim 3, wherein the proton pump inhibitor comprising units are protected by an enteric coating layer and the unit comprising acetyl salicylic acid or a derivative thereof is compressed to a tablet.

5. A dosage form according to claim 4, wherein the unit comprising acetyl salicylic acid or a derivative thereof is mildly compressed to a plug.

6. A dosage form according to claim 5, wherein the plug of ASA has a friability in the range of 2%-50% (w/w).

7. A dosage form according to any of claims 1-6, wherein said dosage form is a capsule formulation or a sachet formulation.

8. A dosage form according to any of claims 1-3, wherein said dosage form is a multiple unit tablet formulation.

9. A dosage form according to any of claims 1-8, wherein the proton pump inhibitor is protected by two layers, an enteric coating layer and a subcoating layer separating the enteric coating from the proton pump inhibitor.

10. A dosage form according to any one of claims 1-9, wherein the proton pump inhibitor is omeprazole or an alkaline salt thereof.

11. A dosage form according to any one of claims 1-9, wherein the proton pump inhibitor is esomeprazole or an alkaline salt thereof or a hydrated form of any one of them.

12. A dosage form according to any one of claims 1-9, wherein the proton pump inhibitor is lansoprazole or a pharmaceutically acceptable salt thereof or a single enantiomer of any one of them.

13. A dosage form according to any one of claims 1-9, wherein the proton pump inhibitor is pantoprazole or a pharmaceutically acceptable salt thereof or a single enantiomer of any one of them.

14. A dosage form according to any one of claims 1-9, wherein the proton pump inhibitor is rabeprazole or a pharmaceutically acceptable salt thereof or a single enantiomer of either one of them.

15. A dosage form according to any one of claims 1-9, wherein the proton pump inhibitor is ilaprazole or a pharmaceutically acceptable salt thereof or a single enantiomer of either one of them.

16. A dosage form according to any one of claims 1-9, wherein the proton pump inhibitor is tenatoprazole or a pharmaceutically acceptable salt thereof or a single enantiomer of either one of them.

17. A dosage form according to any one of claims 1-16, wherein the amount of proton pump inhibitor is in the range of from 5 to 300 mg and the amount of acetyl salicylic acid is in the range of from 10 to 500 mg.

18. A dosage form according to any one of claims 1-17, wherein the amount of proton pump inhibitor is in the range of from 10 to 200 mg.

19. A dosage form according to any one of claims 1-18, wherein the amount of proton pump inhibitor is selected from 5, 10, 20, 30, 40, 50, 60, 70, 80, 90 and 100 mg.

20. A dosage form according to claim any one of claims 1-19, wherein the amount of acetyl salicylic acid is in the range of from 25 to 450 mg.

21. A dosage form according to any one of claims 1-20, wherein the amount of acetyl salicylic acid is in the range of from 50 to 400.

22. A dosage form according to any one of claims 1-21, wherein the amount of acetyl salicylic acid is in the range of from 60 to 350 mg.

23. A dosage form according to any one of claims 1-22, wherein the amount of acetyl salicylic acid is in the range of from 75 to 325 mg.

24. A process for the manufacture of an oral fixed combination dosage form comprising an acid susceptible proton pump inhibitor and acetyl salicylic acid, characterized in that said proton pump inhibitor is prepared in the form of enteric coating layered units and that the units are filled into a capsule or a sachet together with one or more other separate physical units comprising acetyl salicylic acid optionally mixed with pharmaceutically acceptable excipients.

25. A method for the prevention of thromboembolic vascular events, such as myocardial infarction or stroke, and the reduction and/or prevention of gastrointestinal complications associated with acetyl salicylic acid treatment in mammals or man by administering to a host in need thereof a therapeutically effective dose of a fixed dosage form according to any of claims 1 to 23.

26. A method according to claim 25, wherein said method comprises administration of a capsule or a sachet comprising acetyl salicylic acid and proton pump inhibitor.

27. A method according to claim 26, wherein the capsule or sachet is administered once daily.

28. A method according to claim 26, wherein the capsule or sachet is administered twice daily.

29. Use of a dosage form according to any one of claims 1-23, for the manufacture of a medicament for the prevention of thromboembolic vascular events, such as myocardial infarction or stroke, and for the prevention and/or reduction of gastrointestinal complications associated with acetyl salicylic acid treatment.

30. A pharmaceutical oral fixed combination dosage form comprising esomeprazole or an alkaline salt thereof or a hydrated form of any one of them and acetyl salicylic acid, which dosage form is comprising a group of separate physical units comprising the acid susceptible proton pump inhibitor and one or more other separate physical units comprising the acetyl salicylic acid or a derivative thereof, and wherein at least the proton pump inhibitor is protected by an enteric coating layer, wherein said dosage form is for the prevention of thromboembolic vascular events, such as myocardial infarction or stroke, and for the prevention and/or reduction of gastrointestinal complications associated with acetyl salicylic acid treatment.

31. A dosage form according to claim 30, wherein the unit comprising the acetyl salicylic acid or a derivative thereof is compressed, wherein said dosage form is for the prevention of thromboembolic vascular events, such as myocardial infarction or stroke, and for the prevention and/or reduction of gastrointestinal complications associated with acetyl salicylic acid treatment.

32. A dosage form according to claim 30 or 31, wherein the amount of esomeprazole or an alkaline salt thereof or a hydrated form of any one of them is in the range of from 5 to 300 mg and the amount of acetyl salicylic acid is in the range of from 10 to 500 mg.

33. A dosage form according to anyone of claims 30 to 32, which comprises 20 mg of esomeprazole and 325 mg of acetyl salicylic acid.

34. A dosage form according to anyone of claims 30 to 32, which comprises 20 mg of esomeprazole and 75 mg of acetyl salicylic acid.

35. A dosage form according to anyone of claims 30 to 32, which comprises 40 mg of esomeprazole and 325 mg of acetyl salicylic acid.

36. A dosage form according to anyone of claims 30 to 32, which comprises 40 mg of esomeprazole and 75 mg of acetyl salicylic acid.

37. A dosage form according to anyone of claims 30 to 32, which comprises 20 mg of esomeprazole and 81 mg of acetyl salicylic acid.

38. A dosage form according to anyone of claims 30 to 32, which comprises 40 mg of esomeprazole and 81 mg of acetyl salicylic acid.

39. Use of a dosage form according to any one of claims 30-38 for the manufacture of a medicament for administration to a mammal or man, wherein said medicament is for the prevention of thromboembolic vascular events, such as myocardial infarction or stroke, and for the prevention and/or reduction of gastrointestinal complications associated with acetyl salicylic acid treatment.

40. A method for the prevention of thromboembolic vascular events, such as myocardial infarction or stroke, and for prevention and/or reduction of gastrointestinal to complications associated with acetyl salicylic acid treatment in mammal or man by administration to a mammal or man in need thereof a therapeutically effective dose of a combination according to any one of claims 30-38.
